(19) United States
(12) Patent Application Publication
(10) Pub. No.: US 2009/0005699 A1
(43) Pub. Date: Jan. 1, 2009

(54) Input system and computer readable recording medium

(75) Inventors: Satoshi Sakurai, Shinagawa (JP); Shigemi Kurashima, Shinagawa (JP); Takashi Yuba, Shinagawa (JP); Takuya Uchiyama, Shinagawa (JP); Masahiro Yanagi, Shinagawa (JP); Takashi Arita, Shinagawa (JP)

Correspondence Address: STAAS & HALSEY LLP, SUITE 700, 1201 NEW YORK AVENUE, N.W., WASHINGTON, DC, 20005, US

(73) Assignee: FUJITSU COMPONENT LIMITED, Tokyo (JP)

(21) Appl. No.: 12/155,553

(22) Filed: Jun. 5, 2008

(30) Foreign Application Priority Data
Jun. 7, 2007 (JP) 2007-152009

(51) Int. Cl. A61B 5/04

(52) U.S. Cl. 600/546

(57) ABSTRACT

An input system executes an input to an information processing apparatus depending on the hand motion of a person. At least one myoelectric sensor is provided on an area between a wrist of the person and bases of a second finger to a fifth finger, and detects a myoelectric signal depending on the hand motion. A standard value obtaining portion outputs a command to make the person maintain a hand in a constant posture in a state where the myoelectric sensors is worn on the hand, and obtains a value based on the myoelectric signal detected by the myoelectric sensor after the output of the command, as a standard value. A calibration portion calibrates a myoelectric signal depending on the hand motion after the standard value is obtained by the standard value obtaining portion, with the standard value.

### BACKGROUND OF THE INVENTION

1. Field of the Invention

The present invention relates to an input system and a computer readable recording medium, and more particularly to an input system inputting data to an information processing apparatus depending on the hand motion of a person, and a computer readable recording medium which calibrates detection results of myoelectric sensors detecting myoelectric signals depending on the hand motion of a person.

2. Description of the Related Art

Conventionally, there have been known various types of pointing devices such as a mouse, and a track ball, which are used for a pointing operation (an instruction operation) to move a pointer on a screen at a desired position.

Recently, there have been studied a controlling device which detects the motion and the power in each part of a body by using a myoelectric signal detected from a skin-surface electrode, and controls an control object depending on the results of the detection (see e.g. Japanese Laid-Open Patent Publication No. 07-248873), and an input system which decides input information of a keyboard and the like based on the results of the detection of a detecting device such as a myoelectric sensor worn on a finger and the like (see e.g. Japanese Laid-Open Patent Publication No. 07-121294, and Japanese Laid-Open Patent Publication No. 11-338597).

However, in the invention disclosed by Japanese Laid-Open Patent Publication Nos. 07-248873, 07-121294, and 11-338597, the myoelectric signal is acquired (i.e., a myogenic potential occurs) even in a state where the hand and the finger are not moved. Therefore, there is a fear that an error occurs in the results of the detection by the influence of the myoelectric signal.

### SUMMARY OF THE INVENTION

It is an object of the present invention to provide an input system capable of executing an input to an information processing apparatus with high accuracy. It is another object of the present invention to provide a computer readable recording medium capable of calibrating detection results of myoelectric sensors with high accuracy.

According to a first aspect of the present invention, there is provided an input system executing an input to an information processing apparatus depending on the hand motion of a person, including: at least one myoelectric sensor that is provided on an area between a wrist of the person and bases of a second finger to a fifth finger, and detects a myoelectric signal depending on the hand motion; a standard value obtaining portion that outputs a command to make the person maintain a hand in a constant posture in a state where the myoelectric sensors is worn on the hand, and obtains a value based on the myoelectric signal detected by the myoelectric sensor after the output of the command, as a standard value; and a calibration portion that calibrates a myoelectric signal depending on the hand motion after the standard value is obtained by the standard value obtaining portion, with the standard value.

With the above arrangement, the standard value obtaining portion can obtain the myoelectric signal detected by the myoelectric sensor while the person is maintaining the hand in the constant posture in accordance with the command, calibrate a myoelectric signal currently detected by the myoelectric sensor with the standard value (i.e., the myoelectric signal output while the hand is not moving). This makes it possible to detect the calibrated myoelectric signal with high accuracy. As a result, it is capable of executing the input to the information processing apparatus with high accuracy, by using the calibrated myoelectric signal.

Preferably, the standard value is the myoelectric signal detected by the myoelectric sensor at arbitrary time. Since the myoelectric signal while the hand is not moving is used as the standard value, it is possible to detect the calibrated myoelectric signal with high accuracy. As a result, it is capable of executing the input to the information processing apparatus with high accuracy.

Preferably, there are a plurality of myoelectric sensors, and the standard value obtaining portion obtains standard values specific to the respective myoelectric sensors based on the myoelectric signals detected by the myoelectric sensors. In this case, it is possible to reduce a measurement error specific to each of the myoelectric sensors.

More preferably, the standard value obtaining portion obtains myoelectric signals detected by the myoelectric sensors every given time intervals when the hand is maintained in the constant posture, and sets average values of the detected myoelectric signals as the standard values of the myoelectric sensors. In this case, even if a part of the hand is slightly moved while the standard values are obtained, the influence of the movement can be minimized.

According to a second aspect of the present invention, there is provided an input system executing an input to an information processing apparatus depending on the hand motion of a person, including: a plurality of myoelectric sensors that are provided on an area between a wrist of the person and bases of a second finger to a fifth finger, and detect myoelectric signals depending on the hand motion; and a calibration portion that calibrates, with at least one myoelectric signal detected by at least one of the myoelectric sensors as a particular myoelectric sensor, another myoelectric signals detected by another myoelectric sensors.

With the above arrangement, the calibration portion relatively calibrates another myoelectric signals detected by another myoelectric sensors with the myoelectric signal detected by the particular myoelectric sensor. Therefore, highly accurate myoelectric signals that are not affected by the change of environment can be obtained. Further, by inputting the myoelectric signals to the information processing apparatus, highly accurate input can be realized.

Preferably, the particular myoelectric sensor may be a myoelectric sensor in which the fluctuation of the myoelectric signal is the most smallest in the myoelectric sensors, or be worn on a part with the most little motion of the hand. In these case, the calibration is executed by using the myoelectric signal with the most smallest fluctuation, or the myoelectric signal of the myoelectric sensor worn on the part with the most little motion of the hand as a standard value. Therefore, it is possible to detect the calibrated myoelectric signal with high accuracy. As a result, it is capable of executing the input to the information processing apparatus with high accuracy.

According to a third aspect of the present invention, there is provided a computer readable recording medium causing a computer to execute a process, the computer being connected to at least one myoelectric sensor detecting myoelectric signal depending on the hand motion of a person, the process including: a step of outputting a command to make the person maintain a hand in a constant posture; a step of detecting the myoelectric signal depending on the hand motion after the output of the command, and obtaining a value based on the detected myoelectric signal as a standard value; and a step of calibrating a myoelectric signal depending on the hand motion after the standard value is obtained by the standard value obtaining portion, with the standard value.

With the above arrangement, the standard value obtaining portion can obtain the myoelectric signal detected by the myoelectric sensor while the person is maintaining the hand in the constant posture, calibrate a myoelectric signal currently detected by the myoelectric sensor with the standard value (i.e., the myoelectric signal output while the hand is not moving). This makes it possible to detect the calibrated myoelectric signal with high accuracy. As a result, it is capable of executing the input to the information processing apparatus with high accuracy, by using the calibrated myoelectric signal.

According to a fourth aspect of the present invention, there is provided a computer readable recording medium causing a computer to execute a process, the computer being connected to a plurality of myoelectric sensors detecting myoelectric signals depending on the hand motion of a person, the process including: a step of selecting at least one of the myoelectric sensors as a particular myoelectric sensor; and a step of calibrating, with a myoelectric signal detected by the particular myoelectric sensor, another myoelectric signals detected by another myoelectric sensors.

With the above arrangement, the calibration portion relatively calibrates another myoelectric signals detected by another myoelectric sensors with the myoelectric signal detected by the particular myoelectric sensor. Therefore, highly accurate myoelectric signals that are not affected by the change of environment can be obtained. Further, by inputting the myoelectric signals to the information processing apparatus, highly accurate input can be realized.

### BRIEF DESCRIPTION OF THE DRAWINGS

Preferred embodiments of the present invention will be described in detail with reference to the following drawings, wherein:

FIG. 1 is a block diagram showing the construction of an information processing system according to an embodiment of the present invention;

FIG. 2 is a diagram showing a state where a pointing device is worn on a wrist;

FIG. 3 is an exploded perspective view of the pointing device;

FIG. 4 is a diagram showing muscles in the vicinity of the wrist;

FIG. 5 is a flowchart showing a process of the pointing device according to the embodiment of the present invention;

FIG. 6 is a flowchart showing the details of a procedure in step S12 of FIG. 5;

FIG. 7 is a flowchart showing the details of a procedure in step S14 of FIG. 5;

FIG. 8A is a diagram showing a correspondence relationship between pointer moving patterns and pointer motion messages;

FIG. 8B is a diagram showing a correspondence relationship between pointer moving patterns and pieces of characteristic master data;

FIG. 9 is a flowchart showing the details of a procedure in step S16 of FIG. 5;

FIG. 10 is a diagram showing an example of wearing the pointing device according to a variation of the embodiment of the present invention; and

FIG. 11 is a diagram showing muscles in a palm side of the hand.

### DESCRIPTION OF THE PREFERRED EMBODIMENTS

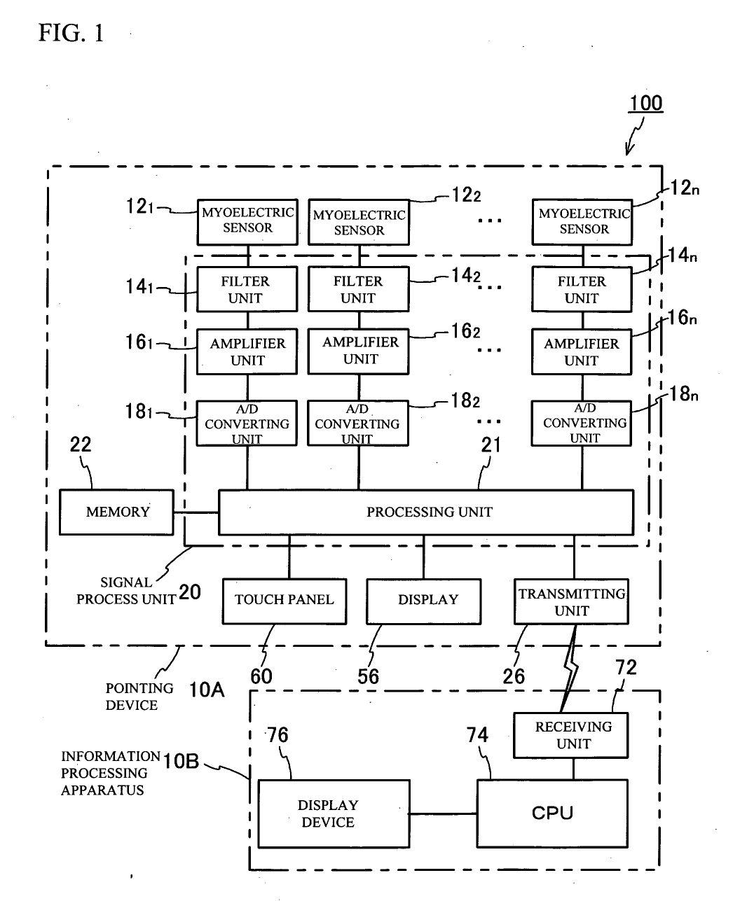

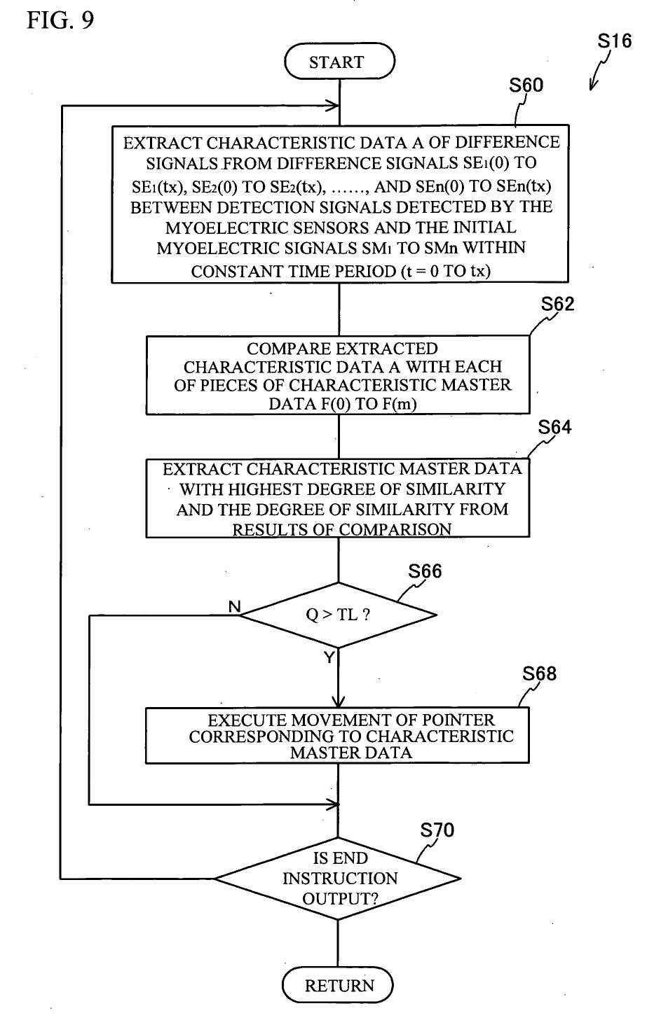

An embodiment of the present invention will be circumstantially described below based on FIGS. 1 to 9.

As shown in a block diagram of FIG. 1, a information processing system 100 according to the embodiment of the present invention is provided with a pointing device 10A which is worn by an operator (a user), and an information processing apparatus 10B which receives the results of a process executed by the pointing device 10A (i.e., the results of the output from the pointing device 10A), and moves a pointer displayed on a display device 76 depending on the results of the process.

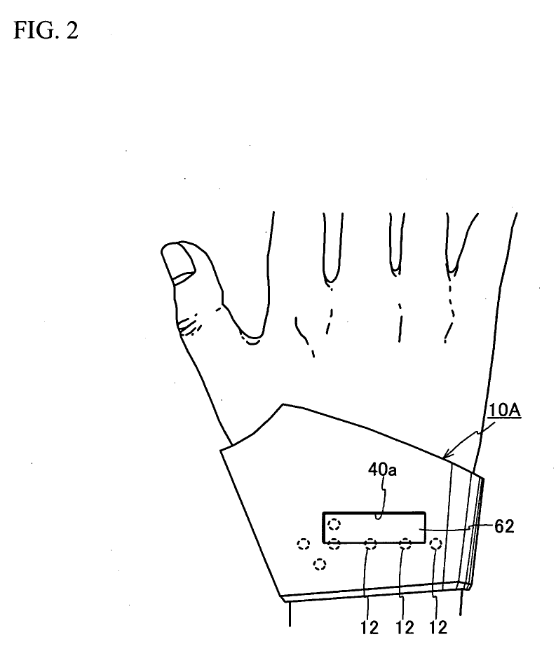

Actually, the pointing device 10A is worn on a part of a wrist of the operator as shown in FIG. 2.

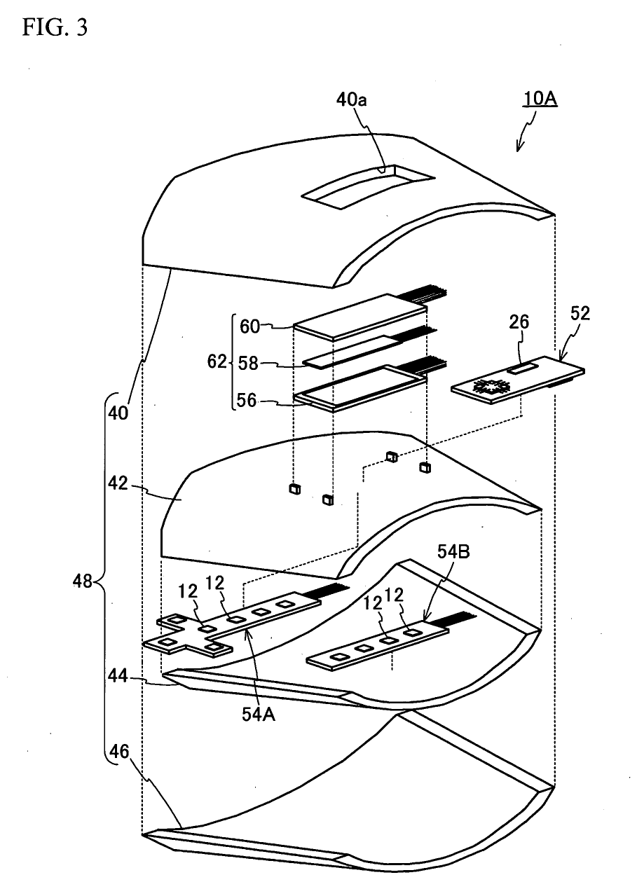

The pointing device 10A is provided with a main body unit 48, a main substrate 52, first and second flexible substrates 54A and 54B each having plural myoelectric sensors 12, and a display unit 62 including a display 56, a transparent solar battery 58, and a touch panel 60, as shown in an exploded perspective view of FIG. 3.

The main body unit 48 includes a first top case 40 where a rectangular window 40a is formed in a center part thereof; a first bottom case 46 having a shape which is in a substantially vertically symmetrical relationship with the first top case 40, and being substantially shaped in a form of annulus by coupling with the first top case 40; a second top case 42 which is provided inside the first top case 40 (i.e., at a bottom side of the first top case 40), and is one size smaller than the first top case 40; and a second bottom case 44 having a shape which is in a substantially vertically symmetrical relationship with the second top case 42, and being substantially shaped in a form of annulus by coupling with the second top case 42. The operator wears the pointing device 10A on the wrist inserted in a space between the second top case 42 and the second bottom case 44 of the main body unit 48. Material having a property of being transformable somewhat, e.g. resin, rubber, or the like, can be adopted as the material of the main body unit 48 to permit the motion of the hand and the wrist. An adjusting mechanism (an adjuster), not shown, for coupling the first top case 40 with the first bottom case 46 and making the main body unit 48 fit the wrist of the operator is provided between the first top case 40 and the first bottom case 46.

The main substrate 52 includes a signal process unit 20 (not shown in FIG. 3; see FIG. 1), a transmitting unit 26, a memory 22 (not shown in FIG. 3; see FIG. 1), a button battery (not shown), and the like, and is provided between the first top case 40 and the second top case 42. A concrete content of the function of each element included in the main substrate 52 will be described later.

On the first and second flexible substrates 54A and 54B, the plural myoelectric sensors 12 (i.e., n myoelectric sensors) are provided at predetermined intervals. In FIG. 1, the plural myoelectric sensors 12 to which subscripts such as “12₁, 12₂ . . . 12ₙ” are added are shown for convenience of explanation. The plural myoelectric sensors 12₁ to 12ₙ detect the myoelectric signals generated by the muscles depending on the motion of the wrist, or the like. Each myoelectric signal changes by the activity of muscular cells, and the amplitude of the change changes in proportion to the size of the muscle activity.

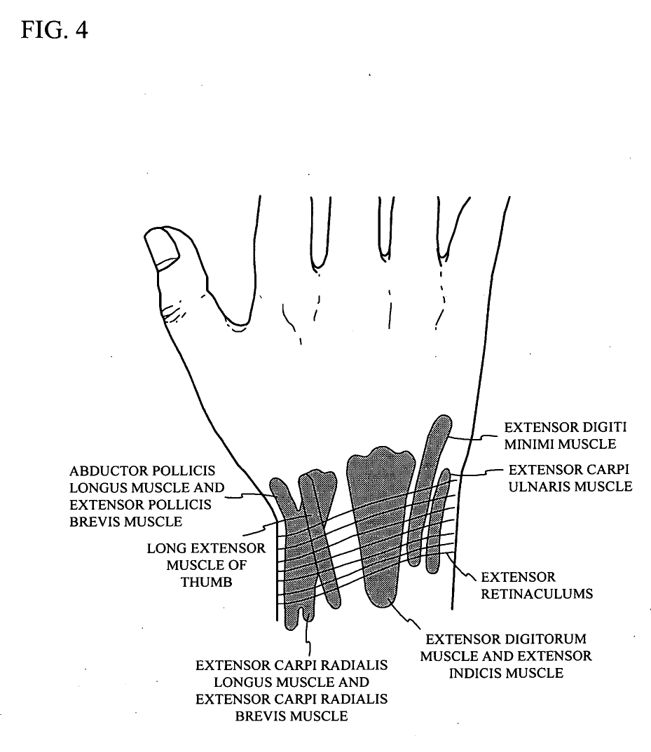

The first flexible substrate 54A is provided inside the second top case 42 (i.e., at a bottom surface of the second top case 42), and the second flexible substrate 54B is provided inside the second bottom case 44 (i.e., at an upper surface of the second bottom case 44). Each of the myoelectric sensors 12 touches the skin in the vicinity of the wrist of the operator. That is, in the embodiment of the present invention, the pointing device 10A is worn on the part of the wrist of the operator as shown in FIG. 2, and the myoelectric sensors 12₁ to 12ₙ can be arranged near the muscles (i.e., an abductor pollicis longus muscle and an extensor pollicis brevis muscle, an extensor carpi radialis longus muscle and an extensor carpi radialis brevis muscle, a long extensor muscle of thumb, an extensor digitorum muscle and an extensor indicis muscle, extensor retinaculums, an extensor carpi ulnaris muscle, an extensor digiti minimi muscle, and the like) which are positioned in the vicinity of the wrist and relate to the finger motion, as shown in FIG. 4. Thus, it is possible to effectively obtain the myoelectric signals of the muscles relating to the finger motion.

Referring to FIG. 3, the display 56 constituting the display unit 62 is composed of an electronic paper, an organic light emitting display, or a liquid crystal display, for example. The transparent solar battery 58 obtains the electric power used for operating the display 56, the touch panel 60, and the like. The touch panel 60 is an input interface for the operator to touch a character, a figure, and so on displayed on the display 56, and to enable the operation of the pointing device 10A. The display unit 62 constituted as above is provided outside the second top case 42 (i.e., at the upper side of the second top case 42) and is in a condition to have been fit in the rectangular window 40a formed on the first top case 40.

Next, the above-mentioned signal process unit 20 will be described below based on FIG. 1. The signal process unit 20 includes filter units 14₁ to 14ₙ corresponding to the n myoelectric sensors (12₁ to 12ₙ), respectively, amplifier units 16₁ to 16ₙ that amplify the myoelectric signals via the filter units 14₁ to 14ₙ, A/D converting units 18₁ to 18ₙ that convert the myoelectric signals (analog signals) via the amplifier units 16₁ to 16ₙ into digital signals, a processing unit 21 as a standard value obtaining portion and a calibration portion of the present invention that processes the output from the A/D converting units 18₁ to 18ₙ.

The filter units 14₁ to 14ₙ are composed of band pass filers having passbands of several tens Hz to 1.5 kHz, for example, and eliminates a polarization voltage of the electrode, a noise of a power supply, a high frequency noise, and the like. The amplifier units 16₁ to 16ₙ amplify the myoelectric signals (ordinarily, about several tens mV) output from the filter units 14₁ to 14ₙ to a level in which signal analysis can be executed. The processing unit 21 processes the digital signals output from the A/D converting units 18₁ to 18ₙ. The memory 22 that stores data used for the process of the digital signals, the display 56 and the touch panel 60 that constitute the display unit 62, and the transmitting unit 26 that transmits the results of the process to a receiving unit 72 in the information processing apparatus 10B are connected to the processing unit 21. Wireless communication using, for example, an electric wave, infrared rays, or the like is executed between the transmitting unit 26 and the receiving unit 72.

The information processing apparatus 10B in FIG. 1 is provided with a CPU 74, the receiving unit 72 that is connected to the CPU 74, and a display device 76 that is composed of a liquid crystal display, a CRT display, a projection system including a projector and a screen, or the like. The CPU 74 moves the pointer displayed on the display device 76 based on the results of the process of the pointing device 10A received by the receiving unit 72.

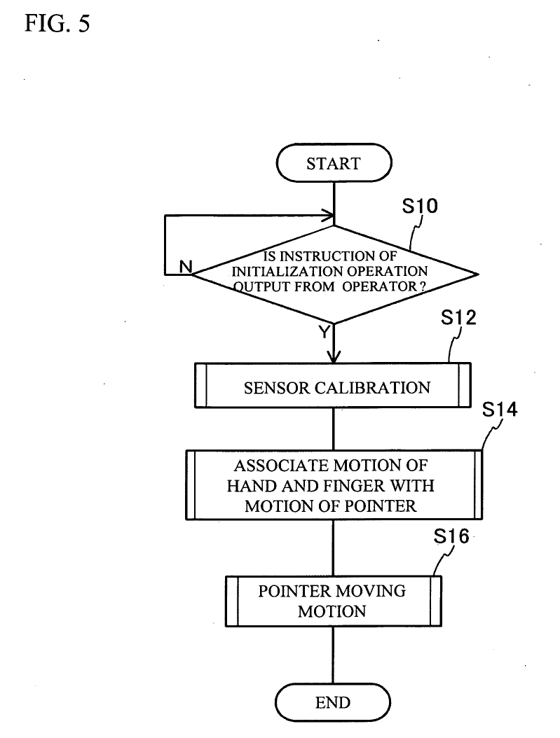

Next, an input process to the information processing apparatus 10B with the pointing device 10A according to the embodiment of the present invention will be described below based on FIGS. 5 to 9.

As a precondition for the input process, a power supply of the information processing apparatus 10B is turned on in advance, and the operator wears the pointing device 10A in the vicinity of the wrist as shown in FIG. 2. In this case, the operator may fit the pointing device 10A to the shape of the wrist to position it at a suitable position, and fix the pointing device 10A on the wrist with the adjusting mechanism (the adjuster), not shown. The following process is mainly executed by the processing unit 21 in the pointing device 10A, and the description of the subject of the process therefore is omitted except a necessary case.

First, in step S10 of FIG. 5, the processing unit 21 waits until an instruction of initialization operation is output from the operator via the touch panel 60 provided on the pointing device 10A. When the instruction of initialization operation is output from the operator via the touch panel 60, the procedure exceeds to next step S12.

In step S12, the processing unit 21 executes a subroutine of a sensor calibration. The subroutine of the sensor calibration is executed to obtain output signals of all the myoelectric sensors 12₁ to 12ₙ in a state where the hand and the finger are maintained in a certain constant posture.

Specifically, the following process is executed.

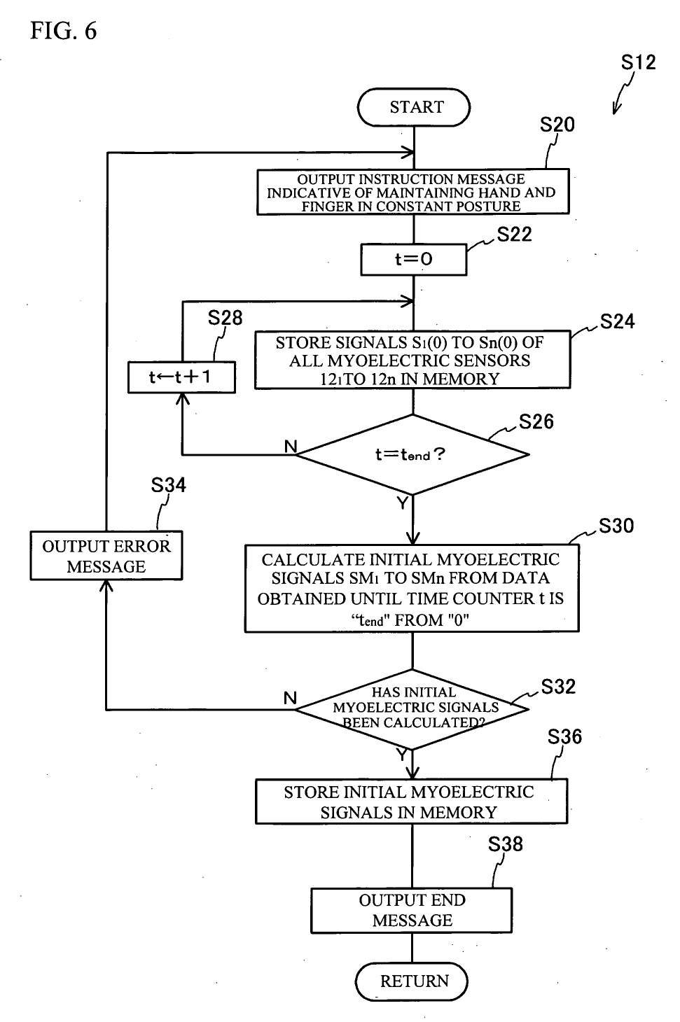

First, as shown in step S20 of FIG. 6, the processing unit 21 outputs an instruction message indicative of maintaining the hand and the finger in a constant posture to the display 56 on the pointing device 10A. Next, in step S22, the processing unit 21 sets a counter t representing time to “0”. In step S24, the processing unit 21 obtains detection signals S₁(0) to Sₙ(0) of all the myoelectric sensors 12₁ to 12ₙ (i.e., digital signals generated via the filter units, the amplifier units, and A/D converting units), and stores the detection signals S₁(0) to Sₙ(0) in the memory 22.

In next step S26, the processing unit 21 determines whether the counter t is t_{end} (the t_{end} represents end time of the subroutine of the sensor calibration). Here, since the counter t is “0”, the answer to the determination of step S26 is “NO”, and the procedure exceeds to step S28. In step S28, the counter t is incremented by 1 (t←t+1), and in step S24, the processing unit 21 then obtains detection signals S₁(1) to Sₙ(1) of all the myoelectric sensors 12₁ to 12ₙ (i.e., digital signals generated via the filter units, the amplifier units, and A/D converting units), and stores the detection signals S₁(1) to Sₙ(1) in the memory 22. Then, the procedures of step S24, step S26, and step S28 are repeated until the counter t is t_{end}, so that the detection signals S₁(0) to S₁(t_{end}), S₂(0) to S₂(t_{end}), . . . , and Sₙ(0) to Sₙ(t_{end}) are stored in the memory 22.

In next step S30, the processing unit 21 calculates initial myoelectric signals SM₁ to SMₙ from the detection signals (data) obtained by the above-mentioned procedures. Here, a mean value of the detection signals S₁(0) to S₁(t_{end}) can be adopted as the initial myoelectric signal SM₁, for example. In this case, the initial myoelectric signal SM₁ is calculated according to the following equation. (1):

$$S {M}_{1} = \frac{1}{{t}_{end}} {\sum }_{t = 0}^{{t}_{end}} {S}_{1} ( t ) \qquad ( 1 )$$

The other initial myoelectric signals SM₂ to SMₙ can be also calculated in a manner as described above.

It should be noted that a decision method of the initial myoelectric signal is not limited to this. Detection signals S₁(tₖ), S₂(tₖ), . . . , and Sₙ(tₖ) with regard to arbitrary time tₖ may be set to the initial myoelectric signals SM₁ to SMₙ, respectively. Moreover, the calculation results obtained by other operations may be set to the initial myoelectric signals SM₁ to SMₙ.

Next, in step S32, the processing unit 21 determines whether the initial myoelectric signals SM₁ to SMₙ have been calculated. Specifically, the processing unit 21 determines whether a signal greatly different from other initial myoelectric signals exists in the initial myoelectric signals calculated in step S30. In the determination of step S32, in the case where the signal greatly different from other initial myoelectric signals exists (i.e., in the case where the operator do not maintain the hand and the finger in the constant posture, and right initial myoelectric signals are not obtained) is regarded as in the case where the initial myoelectric signals are not calculated. When the answer to the determination of step S32 is “NO” (i.e., when the initial myoelectric signals are not calculated), the processing unit 21 outputs (displays) an error message to the display 56 in step S34 to execute the procedures of steps S20 to S30 again. The procedure exceeds to step S20, and the processing unit 21 outputs the instruction message indicative of maintaining the hand and the finger in the constant posture to the display 56 again. Then, the procedures of steps S20 to S34 are repeated until the answer to the determination of step S32 is “YES”.

Then, when the answer to the determination of step S32 is “YES”, the processing unit 21 stores the initial myoelectric signals SM₁ to SMₙ calculated in step S30 in the memory 22, in step S36. In step S38, the processing unit 21 outputs (displays) to the display 56 an end message to tell the end of the subroutine of the sensor calibration to the operator, and terminates the subroutine of the sensor calibration in FIG. 6. The procedure exceeds to step S14 of FIG. 5.

Next, in step S14 of FIG. 5, the processing unit 21 associates the motion of the hand and the finger with the motion of the pointer (hereinafter referred to as “the association subroutine”).

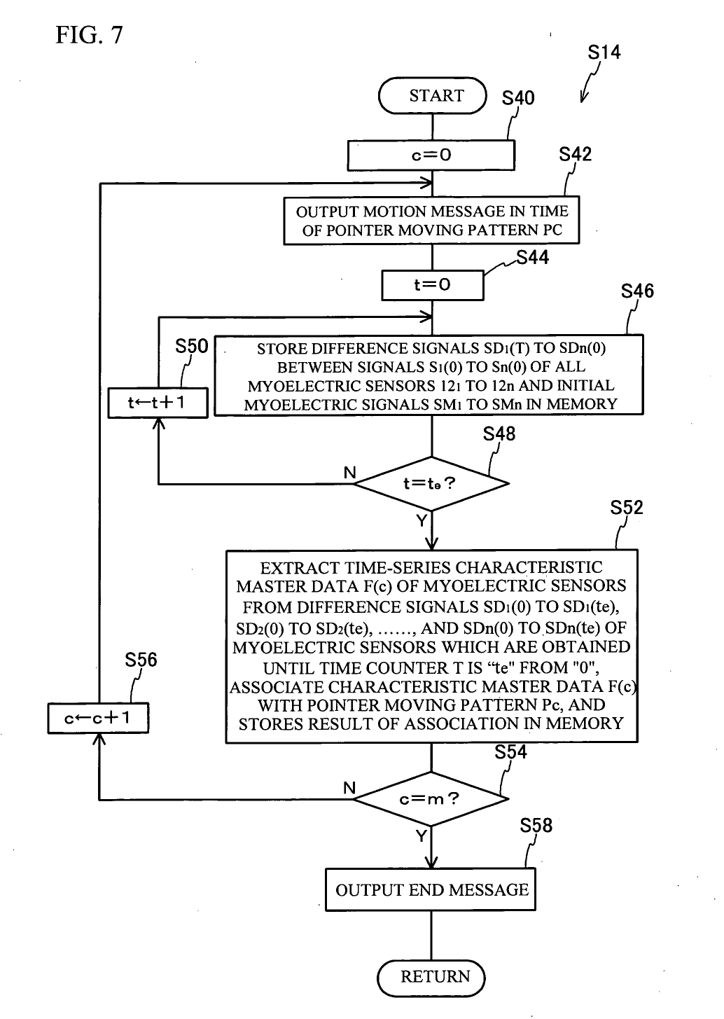

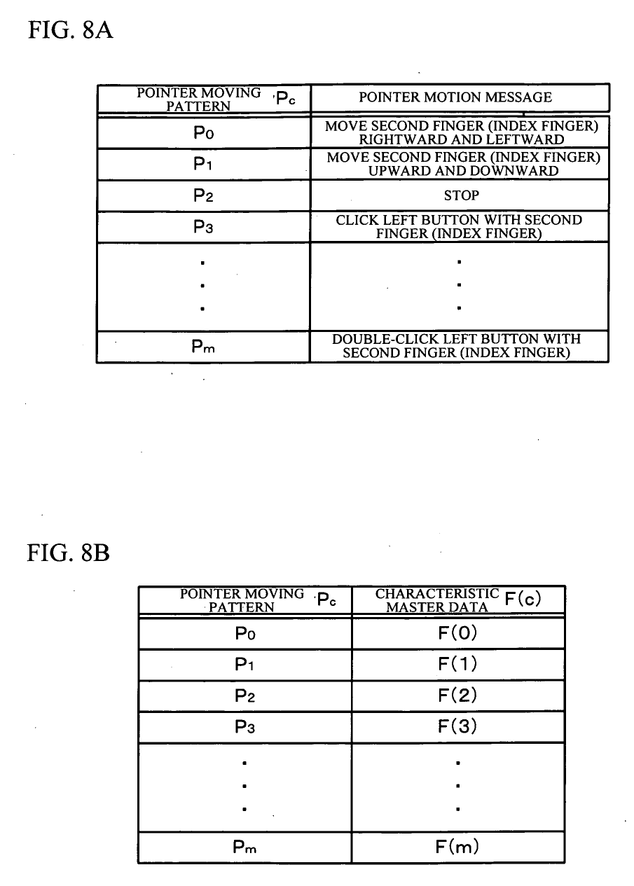

In the association subroutine, the processing unit 21 first sets a counter c of a pointer moving pattern to “0” in step S40 of FIG. 7. Next, in step S42, the processing unit 21 displays a motion message when the pointer moving pattern P_{c} is “P₀”. In this case, a pointer motion message is set as shown in a table of FIG. 8A. Here, the processing unit 21 therefore outputs (displays) the motion message indicative of moving a second finger (i.e., an index finger) rightward and leftward to the display 56 on the pointing device 10A.

In next step S44, the processing unit 21 sets a time counter t to “0”. In next step S46, the processing unit 21 obtains detection signals S₁(0) to Sₙ(0) of all the myoelectric sensors 12₁ to 12ₙ in a state where the operator moves the second finger (i.e., the index finger) rightward and leftward, and reads the initial myoelectric signals SM₁ to SMₙ obtained by the subroutine of the sensor calibration (step S12) from the memory 22. Then, the processing unit 21 calculates difference signals (SD₁(0) to SDₙ(0)) according to the following equation (2), and stores the difference signals in the memory 22:

$$\begin{matrix}S {D}_{1} ( 0 ) = {S}_{1} ( 0 ) - S {M}_{1} \\ S {D}_{2} ( 0 ) = {S}_{2} ( 0 ) - S {M}_{2} \\ ⋮ \\ S {D}_{n} ( 0 ) = {S}_{n} ( 0 ) - S {M}_{n}\end{matrix} \qquad ( 2 )$$

Next, in step S48, the processing unit 21 determines whether the time counter t is tₑ (the tₑ represents end time of the association subroutine). Here, since the time counter t is “0”, the answer to the determination of step S48 is “NO”, and the procedure exceeds to step S50. In step S50, the time counter t is incremented by 1 (t←t+1), and in step S46, the processing unit 21 then obtains detection signals S₁(1) to Sₙ(1) of all the myoelectric sensors 12₁ to 12ₙ, calculates difference signals SD₁(1) to SDₙ(1) indicative of the differences between the detection signals S₁(1) to Sₙ(1) and the initial myoelectric signals SM₁ to SMₙ in the same manner as the above-mentioned equation (2), and stores the difference signals SD₁(1) to SDₙ(1) in the memory 22. Then, the procedures of step S46, step S48, and step S50 are repeated until the time counter t is tₑ, so that the difference signals SD₁(0) to SD₁(tₑ), SD₂(0) to SD₂(tₑ), . . . , and SDₙ(0) to SDₙ(tₑ) are stored in the memory 22.

Next, in step S52, the processing unit 21 extracts time-series characteristic master data F(c) of the myoelectric sensors 12₁ to 12ₙ from the difference signals SD₁(0) to SD₁(tₑ), SD₂(0) to SD₂(tₑ), . . . , and SDₙ(0) to SDₙ(tₑ) of the myoelectric sensors 12₁ to 12ₙ which are obtained until the time counter t is “tₑ” from “0”, associates characteristic master data F(c) (here, c=0) with the pointer moving pattern P₀, and stores the result of the association in the memory 22. In this case, an integrated value average voltage (IEMG), an average frequency (MPF), a center frequency, a root-mean-square value (RMS), a standard deviation of frequency distribution (SDFD), a frequency spectrum, or the like can be used as the characteristic master data.

In next step S54, the processing unit 21 determines whether the counter c is “m” (“m” shows the number of all pointer moving patterns). Here, since the counter c is “0”, the answer to the determination of step S54 is “NO”, and the procedure exceeds to step S56. In step S56, the counter c is incremented by 1 (c←c+1), and then the procedure returns to step S42. Then, the procedures of step S42 to step S56 are repeated in a manner as described above, so that the processing unit 21 associates pieces of characteristic master data F(c) and all the pointer moving patterns P_{c} with each other. FIG. 8B shows a table indicative of a state where all the pointer moving patterns P_{c} and the pieces of characteristic master data F(c) are associated with each other.

Then, the association between all the pointer moving patterns P_{c} and the pieces of characteristic master data F(c) is terminated. When the answer to the determination of step S54 is “YES”, the procedure exceeds to step S58. In step S58, the processing unit 21 outputs (displays) an end message to the display 56, and terminates the association subroutine in FIG. 7. Then, the procedure returns to step S16 of FIG. 5.

In step S16 of FIG. 5 (i.e., a subroutine of a pointer moving motion), the motion of the operator is identified by using data stored in the memory 22 in steps S12 and S14, the movement of the pointer corresponding to the motion of the operator is executed.

In the subroutine of the pointer moving motion, the processing unit 21 first extracts characteristic data A of the difference signals from the difference signals SE₁(0) to SE₁(tₓ), SE₂(0) to SE₂(tₓ), . . . , and SEₙ(0) to SEₙ(tₓ) between the detection signals detected by the myoelectric sensors 12₁ to 12ₙ and the initial myoelectric signals SM₁ to SMₙ within a constant time period (t=0 to tₓ) in step S60 of FIG. 6. This procedure is similar to the above-mentioned procedures of steps S46 and S52 of FIG. 7. In this case, it is required that the characteristic data A is the same type as the characteristic master data F(c) extracted in the above-mentioned procedure of step S52.

In next step S62, the processing unit 21 compares the extracted characteristic data A with each of the pieces of characteristic master data F(0) to F(m). Then, in next step S64, the processing unit 21 extracts characteristic master data with the highest degree of similarity (i.e., any one of F(0) to F(m)) and the degree of similarity Q from the results of the comparison in step S62.

Further, in step S66, the processing unit 21 determines whether the degree of similarity Q is larger than a preset threshold value TL (i.e., a threshold level). In this case, if the degree of similarity Q is larger than the preset threshold value TL, this means that the motion of the operator is identical with the pointer moving pattern P_{c} corresponding to the characteristic master data with the highest degree of similarity. Therefore, when the answer to the determination of step S66 is “YES” (i.e., Q>TL), the procedure exceeds to step S68. In step S68, the processing unit 21 selects the pointer moving pattern corresponding to the characteristic master data with the highest degree of similarity, and outputs the selected pointer moving pattern as output information to the CPU 74 via the transmitting unit 26 and the receiving unit 72. The CPU 74 controls the pointer according to the output information from the processing unit 21 so that the pointer displayed on the display device 76 is moved according to the pointer moving pattern.

Then, in step S70, the procedures of steps S60 to S68 are repeated until an end instruction is output by the operator via the touch panel 60. When the answer to the determination of step S70 is “YES”, the sequence of the procedures is terminated.

In the above-mentioned subroutine of the pointer moving motion, the operators only moves the hand and the finger, so that the pointer moves according to it. Therefore, when the operators do not want to move the pointer, it is possible to install a mode which can stop the movement of the pointer in the pointing device 10A without removing the pointing device 10A from the wrist. By installing such a mode in the pointing device 10A, when a suspension button is displayed on the display 56 for example, and the operators does not want to move the pointer, it is possible to temporarily stop the procedure of step S16 (i.e., the subroutine of the pointer moving motion) by depressing the suspension button via the touch panel 60.

In the above-mentioned embodiment of the present invention, the difference signals between the actual detection signals of the myoelectric sensors and the initial myoelectric signals are used as the output of the myoelectric sensors 12₁ to 12ₙ (steps S46 and S60). However, the present invention is not limited to this. For example, a difference signal between a detection signal of each of the myoelectric sensors and a detection signal of a particular myoelectric sensor (hereinafter referred to as “myoelectric sensor 12_{b}”) in the myoelectric sensors 12₁ to 12ₙ can be used as the output. In this case, for example, a myoelectric sensor, in which few myoelectric signals are detected for a long time period, in the myoelectric sensors 12₁ to 12ₙ can be adopted as the myoelectric sensor 12_{b}. Specifically, in a design stage of the pointing device 10A, a myoelectric sensor is installed at a position where a myoelectric signal is not detected in the pointing device 10A, and the myoelectric sensor can be set to the myoelectric sensor 12_{b}.

By obtaining the difference signal for which the detection signal of such particular myoelectric sensor 12_{b} is used, even when there is a change of environment during the use of the pointing device 10A, a highly accurate detection result that is not affected by the change of environment can be obtained. Further, by inputting the detection result to the information processing apparatus 10B, highly accurate input (e.g., the movement of the pointer) can be realized. The particular myoelectric sensor is not limited to a single sensor, but may be configured to be comprised of two or more sensors. In this case, a difference signal between a detection signal of the myoelectric sensor and an average of detection signals of the two or more particular myoelectric sensors may be used as the output. Moreover, other operation process is executed to the detection signals of the two or more particular myoelectric sensors, and a difference signal between the detection signal of the myoelectric sensor and the operation result may be used as the output.

As described in detail above, according to the embodiment of the present invention, the processing unit 21 obtains the detection results (i.e., detection signals) of the myoelectric sensors 12₁ to 12ₙ as the initial myoelectric signals SM₁ to SMₙ in a state where the operator maintains the hand in the constant posture according to the instruction (i.e., message), and calibrates actual detection results of the myoelectric sensors 12₁ to 12ₙ with the initial myoelectric signals SM₁ to SMₙ (i.e., calculates the difference signals between the detection signals of the myoelectric sensors 12₁ to 12ₙ and the initial myoelectric signals SM₁ to SMₙ). This makes it possible to detect the myoelectric signals with high accuracy in a state where an error specific to each of the myoelectric sensors is offset. As a result, it is capable of executing the input (e.g., the movement of the pointer) to the information processing apparatus 10B with high accuracy, by using the detected myoelectric signals.

According to the embodiment of the present invention, the processing unit 21 obtains the detection results (i.e., detection signals) of the myoelectric sensors 12₁ to 12ₙ every given time intervals when the hand is maintained in the constant posture, and sets average values of the detection results to the initial myoelectric signals SM₁ to SMₙ. Therefore, even if a part of the hand is slightly moved during the detection of the detection signals, the influence of the movement can be minimized.

According to the embodiment of the present invention, the processing unit 21 obtains the initial myoelectric signals SM₁ to SMₙ specific to the respective myoelectric sensors 12₁ to 12ₙ based on the detection results of the plurality of myoelectric sensors 12₁ to 12ₙ, and it is therefore possible to reduce a measurement error specific to each of the myoelectric sensors 12₁ to 12ₙ.

Although in the embodiment of the present invention, the pointing device 10A has the plurality of myoelectric sensors, the present invention is not limited to this. To obtain the initial myoelectric signals, and calibrate the detection results (i.e., detection signals) of the myoelectric sensors with the obtained initial myoelectric signals, the pointing device 10A may have a single myoelectric sensor.

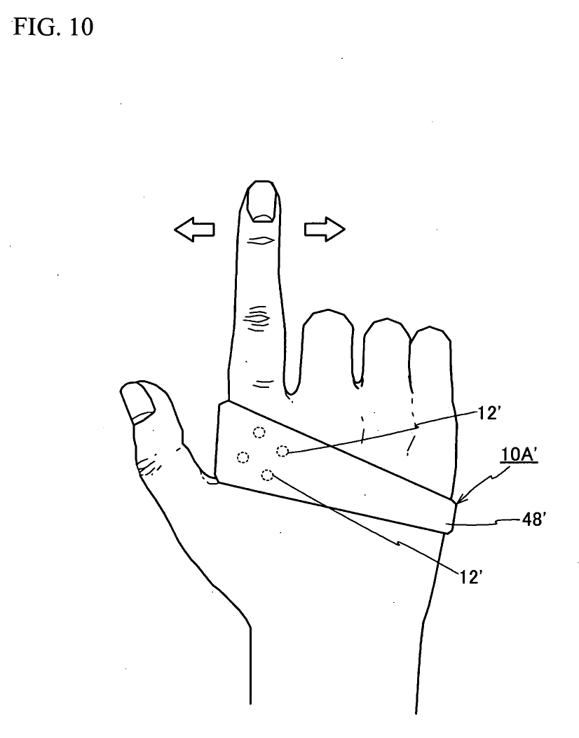

Although in the embodiment of the present invention, the pointing device 10A is worn in the vicinity of the wrist of the operator, the present invention is not limited to this. For example, a pointing device 10A′ as shown in FIG. 10 may be adopted. The pointing device 10A′ is provided with a main body unit 48′ which is shaped in the form of annulus and is worn so as to surround the palm and the back of the hand in a state where the hand of the operator from the 2nd finger (index finger) to a 5th finger (pinky finger) is inserted into the main body unit 48′, plural myoelectric sensors 12′ provided in an inner peripheral surface (a palm side of the hand) of the main body unit 48′.

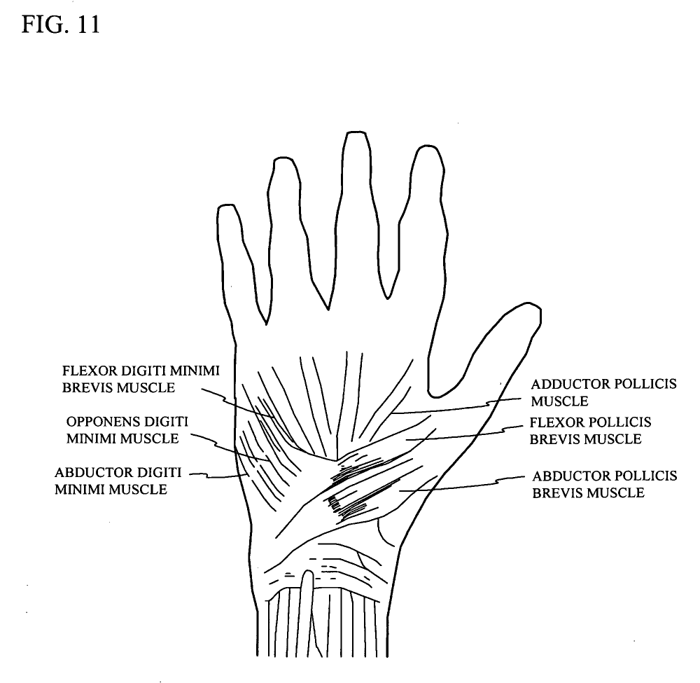

By adopting such pointing device 10A′, the finger motion is not limited as in the case where the main body unit is worn on the finger, and difficulty of the wearing and the possibility of the falling off are also reduced. Moreover, as is apparent from FIG. 11 indicative of muscles on the palm side of the hand, myoelectric sensors 12′ can be made to touch to the muscles on the palm side of the hand, such as a adductor pollicis muscle, a flexor pollicis brevis muscle, an opponens digiti minimi muscle, a flexor digiti minimi brevis muscle, and an abductor digiti minimi muscle, and muscular discharge (an electric potential detected when each muscle shrinks) of each muscle on the palm side of the hand, which shrinks when each finger bends, can be detected. As a result, bending of each finger can be detected in an appropriate timing. It should be noted that, similarly to the above described embodiment, the pointing device 10A′ in FIG. 10 may also be provided with the display 56 or the like. Similarly to the above described embodiment, the pointing device 10A′ may execute the subroutine of the sensor calibration, the association subroutine, and the like, and then execute the pointer moving motion with the results of the subroutines.

Although in the embodiment and the variation of the present invention, the main body unit 48 and the main body unit 48′ are shaped in the form of annulus, the present invention is not limited to this. For example, each of the main body unit 48 and the main body unit 48′ may be substantially shaped in the form of annulus having a cutout at a part thereof. In this case, the width of the cutout is variable by elastic force of the main body unit 48 or the main body unit 48′, and it is therefore possible to finely adjust an inner diameter of the main body unit 48 or the main body unit 48′ according to the size of the hand.

Although in the embodiment and the variation of the present invention, the movement of the pointer on the display device 76 is executed, the present invention is not limited to this. For example, given commands to the motions of the fingers and the hand are preset. When the operator executes any one of the motions of the fingers and the hand, the information processing apparatus 10B may execute given operation (e.g. resume, suspend, power-off, or the like).

Further, the embodiment and the variation of the present invention is not limit to the movement of the pointer. For example, the finger motion is detected, so that the character a character corresponding to the finger motion may be input (keyboard input).

The present invention is not limited to the above embodiment. It should be understood that various changes and modifications may be made without departing from the spirit and scope of the present invention.

The present application is based on Japanese Patent Application No. 2007-152009 filed Jun. 7, 2007, the entire disclosure of which is hereby incorporated by reference.

What is claimed is:

1. An input system executing an input to an information processing apparatus depending on the hand motion of a person, comprising:
at least one myoelectric sensor that is provided on an area between a wrist of the person and bases of a second finger to a fifth finger, and detects a myoelectric signal depending on the hand motion;
a standard value obtaining portion that outputs a command to make the person maintain a hand in a constant posture in a state where the myoelectric sensors is worn on the hand, and obtains a value based on the myoelectric signal detected by the myoelectric sensor after the output of the command, as a standard value; and
a calibration portion that calibrates a myoelectric signal depending on the hand motion after the standard value is obtained by the standard value obtaining portion, with the standard value.

2. The input system as claimed in claim 1, wherein the standard value is the myoelectric signal detected by the myoelectric sensor at arbitrary time.

3. The input system as claimed in claim 1, wherein there are a plurality of myoelectric sensors, and the standard value obtaining portion obtains standard values specific to the respective myoelectric sensors based on the myoelectric signals detected by the myoelectric sensors.

4. The input system as claimed in claim 3, wherein the standard value obtaining portion obtains myoelectric signals detected by the myoelectric sensors every given time intervals when the hand is maintained in the constant posture, and sets average values of the detected myoelectric signals as the standard values of the myoelectric sensors.

5. An input system executing an input to an information processing apparatus depending on the hand motion of a person, comprising:
a plurality of myoelectric sensors that are provided on an area between a wrist of the person and bases of a second finger to a fifth finger, and detect myoelectric signals depending on the hand motion; and
a calibration portion that calibrates, with at least one myoelectric signal detected by at least one of the myoelectric sensors as a particular myoelectric sensor, another myoelectric signals detected by another myoelectric sensors.

6. The input system as claimed in claim 5, wherein the particular myoelectric sensor is a myoelectric sensor in which the fluctuation of the myoelectric signal is the most smallest in the myoelectric sensors.

7. The input system as claimed in claim 5, wherein the particular myoelectric sensor is worn on a part with the most little motion of the hand.

8. A computer readable recording medium causing a computer to execute a process, the computer being connected to at least one myoelectric sensor detecting myoelectric signal depending on the hand motion of a person, the process comprising:
a step of outputting a command to make the person maintain a hand in a constant posture;
a step of detecting the myoelectric signal depending on the hand motion after the output of the command, and obtaining a value based on the detected myoelectric signal as a standard value; and
a step of calibrating a myoelectric signal depending on the hand motion after the standard value is obtained by the standard value obtaining portion, with the standard value.

9. A computer readable recording medium causing a computer to execute a process, the computer being connected to a plurality of myoelectric sensors detecting myoelectric signals depending on the hand motion of a person, the process comprising:
a step of selecting at least one of the myoelectric sensors as a particular myoelectric sensor; and
a step of calibrating, with a myoelectric signal detected by the particular myoelectric sensor, another myoelectric signals detected by another myoelectric sensors.
